(19) United States
(12) Patent Application Publication
(10) Pub. No.: US 2005/0158877 A1
(43) Pub. Date: Jul. 21, 2005

(54) Novel antibody mediated surface enhanced raman scattering (SERS) immunoassay and multiplexing schemes

(76) Inventors: Chengrong Wang, Glen Mills, PA (US); Sean Xiaolu Wang, Centerville, DE (US); Qun Li, Newark, DE (US)

Correspondence Address: BLANK ROME LLP, 600 NEW HAMPSHIRE AVENUE, N.W., WASHINGTON, DC, 20037, US

(21) Appl. No.: 11/029,334

(22) Filed: Jan. 6, 2005

Related U.S. Application Data
(60) Provisional application No. 60/534,371, filed on Jan. 6, 2004.

(51) Int. Cl. G01N 33/551

(52) U.S. Cl. 436/524

(57) ABSTRACT

Assay reagents include small metallic particles labeled with a Raman dye and antibody molecules capable of binding the analyte of interest. In the absence of the analyte, the size of the isolated metallic particles is so small that no significant SERS signal can be detected. An analyte molecule binds two metallic particles together through the formation of a sandwich complex, and then more metallic particles are clustered together by the analyte if each metallic particle contains more than two antibodies on the surface. The bonded metallic particles form a cluster structure, which significantly amplifies the SERS effect.

### REFERENCE TO RELATED APPLICATION

The present application claims the benefit of U.S. Provisional Patent Application No. 60/534,371, filed Jan. 6, 2004, whose disclosure is hereby incorporated by reference in its entirety into the present disclosure.

### FIELD OF THE INVENTION

This invention generally relates to immunoassay and more specifically to antibody mediated surface enhanced Raman scattering (SERS) immunoassay and multiplexing schemes.

### DESCRIPTION OF RELATED ART

Most of the large bio-molecules (the analytes of interest) have two antibody binding sites that will allow the formation of sandwich type complexes, as shown in FIG. 1. The technique for detecting the sandwich formation, which is directly related to the amount of analyte, in many immunoassays determines the assay sensitivity.

The common techniques used today to detect the sandwich formation are radioactive, enzyme or dye (color or fluorescent or chemiluminescent) labeled antibody approaches. However, many of the approaches mentioned above require a physical separation step to remove un-bound labeled antibody to obtain the specific signal.

Surface Enhanced Raman Scattering (SERS) is a signal amplification technique resulting in significant enhancement of Raman signal from molecules which are attached to nano-meter sized metallic particles (especially for gold or silver nano-particles). This technique has the potential for ultra-sensitive trace level chemical analysis or even single molecule detection, as described by K. Kneipp et al. in “Surface-enhanced Raman Scattering and Biophysics”, Journal of Physics: Condensed Matter, Vol. 14, R597-R624, 2002; “Ultrasensitive Chemical Analysis by Raman spectroscopy,” Chemical Reviews, Vol. 99, No.10, 2957-2975, 1999; and “Single Molecule Detection using Surface-Enhanced Raman Scattering (SERS)”, Physics Review Letters, Vol.78, No.9, 1667-1670, 1997. However, to achieve such a high sensitivity, the size, shape and distribution of the metallic nano-particles have to be carefully optimized.

Some examples in the prior art of the application of SERS for immunoassay were disclosed by Tarcha et al. in U.S. Pat. No. 5,266,498, “Ligand binding assay for an analyte using surface-enhanced scattering (SERS) signal,” Nov. 30, 1993; U.S. Pat. No. 5,376,556, “Surface-enhanced Raman spectroscopy immunoassay,” Dec. 27, 1994; and U.S. Pat. No. 5,567,628, “Surface-enhanced Raman spectroscopy immunoassay method, composition and kit,” Oct. 22, 1996. In these patents, colloidal metallic particles in isolated stage are utilized for the SERS. The assay reagents consist of an antibody immobilized gold or silver nano-particle and a Raman dye labeled antibody. In the presence of analyte, the Raman dye labeled antibody will be brought to the gold or silver nano-particle surface to form a sandwich. The proximity effect of the metallic nano-particle will amplify the Raman scattering signal of the dye labeled on the antibody and give the specific Raman scattering signal.

The drawback of the above approach is that the surface enhancement effect induced by the isolated metallic particle is not strong enough, so that the sensitivity of the technique is limited. Similar weakness exists in another variation of the technique as disclosed by White et al. in Published International Patent Application No. WO 99/44065, “Immunoassay involving surface enhanced Raman scattering,” Sep. 02, 1999 and U.S. Pat. No. 6,750,065 B1, “Immunoassay involving surface enhanced Raman scattering,” Jan. 15, 2004, in which displacement SERS immunoassay was discussed.

### SUMMARY OF THE INVENTION

It is an object of the invention to overcome the above deficiencies in the prior art.

It is another object of the invention to eliminate the requirement for a physical separation step.

It is still another object of the invention to increase the sensitivity of SERS.

To achieve the above and other objects, the present invention is directed to a novel antibody mediated SERS immunoassay (AMSERSIA). The AMSERSIA is a new homogenous immunoassay to detect ultra-low levels of bio-molecules. The principle of AMSERSIA is to use the analyte of interest to coagulate Raman dye labeled gold or silver nano-particles to form a cluster structure for strong SERS enhancement.

This invention relates to a novel detection method to determine the presence or amount of analyte of interest in a test sample (biological, environmental or other samples) by monitoring the antibody mediated binding event in the test mixture. The antibody mediated binding event could be the well-known sandwich complex formation (one analyte with two or more antibodies) or two small analyte molecules binding to one single antibody. The antibody mediated binding event (formation or dissociation of the antibody-analyte complex) is then detected.

The present AMSERSIA approach will eliminate the need for a physical separation step during the assay and also enhances the detection sensitivity due to extraordinary amplification effect of SERS. The AMSERSIA technique could also be extended to small analytes via inhibition type assay. While inhibition type assay is well-known by the experts in the art, its use in the context of the present invention is novel.

### BRIEF DESCRIPTION OF THE DRAWINGS

Preferred embodiments of the present invention will be set forth in detail with reference to the drawings, in which:

FIG. 1 illustrates the sandwich type binding of an analyte molecule with two corresponding antibody molecules;

FIG. 2 illustrates the AMSERSIA scheme;

FIG. 3 shows the Raman scattering spectra of two typical cyanine dyes; and

FIG. 4 illustrates the multiplexing scheme for AMSERSIA.

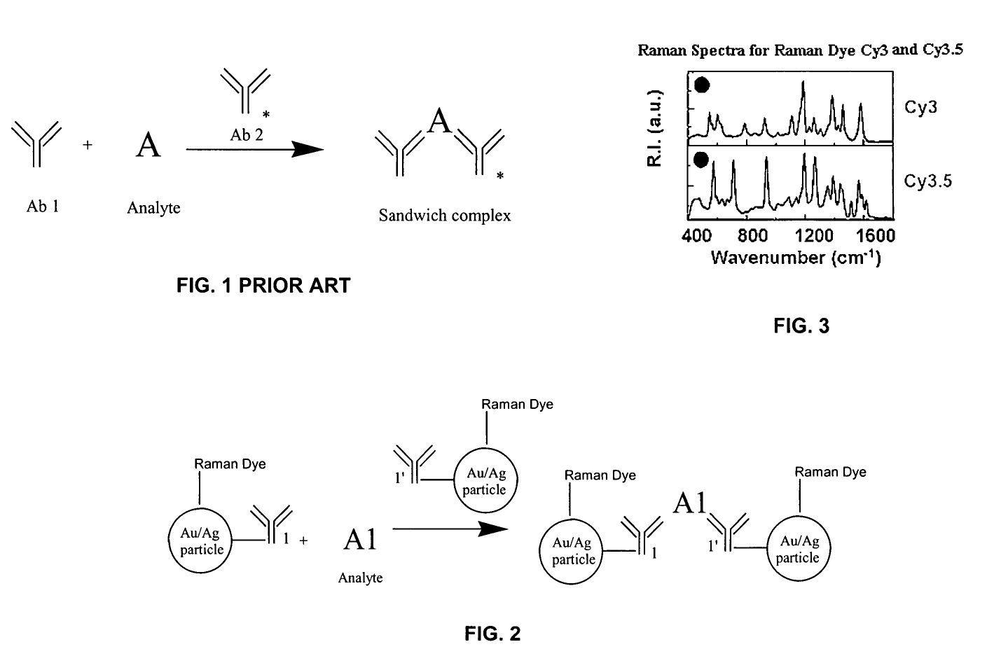

### DETAILED DESCRIPTION OF THE PREFERRED EMBODIMENTS

Preferred embodiments of the present invention will be set forth in detail with reference to the drawings.

The preferred embodiments aim at high sensitivity trace level analyte detection. It utilizes the analyte induced aggregation effect to coagulate the isolated metallic particles to form a cluster structure, which will significantly enhance the SERS effect.

The assay scheme is illustrated schematically in FIG. 2. The assay reagents consist of small metallic particles with size generally smaller than 50 nm. Proper antibody molecules capable of binding the analyte of interest are immobilized on the Raman dye labeled metallic nano-particles. The concentrations of the antibody and metallic colloidal solution are carefully controlled so that each metallic particle accommodates one or more antibody molecules.

In the absence of the analyte, the size of the isolated metallic particles is so small that no significant SERS signal can be detected. When the analyte molecule exists in the solution, it will bind two metallic particles together through the formation of sandwich complex, and then more metallic particles will be clustered together by the analyte if each metallic particle contains more than two antibodies on the surface. The bonded metallic particles form a cluster structure, which significantly amplifies the SERS effect through the following mechanisms. First, the effective surface size of the clustered metallic particles is much larger than that of the isolated particles. Second, there is a strong electromagnetic field enhancement at the midpoint between two closely spaced metallic particles. Third, additional local field enhancement mechanisms arise from sharp edges, kinks or other fractal structures provided by the cluster structure. All these make the clustered metallic particles a “hot spot” for Raman signal generation.

Although theoretical explanation for this cluster SERS effect is still tentative, experimental data as described by K. Kneipp et al. have shown an electromagnetic enhancement factor of 10¹¹-10¹² for the ‘hot spot’ of the cluster structure as compared with the electromagnetic enhancement factor of 10⁶-10⁷ for the isolated metallic particles. Thus, the existence of the analyte molecule can be easily detected through the strong “hot spot” SERS signal.

The Raman signal from the isolated metallic particles is about 10⁵ times weaker than that from the clustered particles. Therefore, there is no need to separate the isolated metallic particles from the assay media during detection. This novel antibody mediated SERS immunoassay does not need any physical separation and washing steps. The extraordinary SERS effect would also allow the detection limit of the assay comparable to a chemiluminescent label based immunoassay, which would have higher assay sensitivity than the one of the fluorescent label based immunoassay in general.

The AMSERSIA can be easily extended to an inhibition type assay for detection of small molecule analytes. In this scheme, analyte analogues are first attached to a metallic particle surface. Then the metallic colloidal solution is mixed with the antibody solution. Each antibody molecule will bind with two analyte analogue molecules, thus causing the metallic particles to aggregate and form a cluster structure for SERS signal amplification. In the presence of analyte, the antibody molecule reacts with the analyte molecule and the formation of the cluster structure is inhibited, which results in a decrease of the Raman signal. Thus the presence and amount of the analyte can be inferred from the intensity variation of the Raman signals.

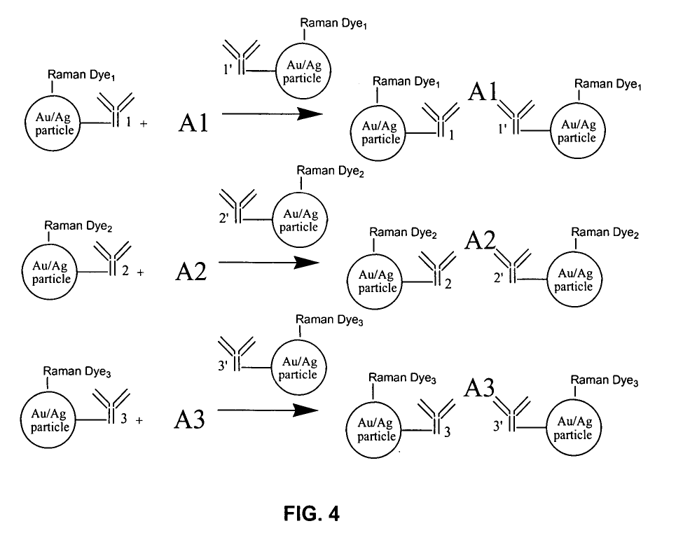

As another aspect of this invention, a novel multiplexing approach for the AMSERSIA is disclosed. It is capable of detecting a panel of bio-molecules at the same time. The multiplexing approach takes advantage of the unique sharpness and narrow linewidth of the Raman scattering peaks to achieve multiple analyte detection. Since different Raman dyes have different Raman scattering peaks and the peaks are sharp and narrow, we can detect several Raman dyes simultaneously by choosing unique Raman scattering peak for each dye. As an example, the Raman scattering spectra of two typical cyanine dyes are shown in the FIG. 3, in which specific peaks can be picked for each dye to determine the concentration. There are many commercially available Raman dyes such as Rhodamine 6G, crystal violet and cyanine dyes (Cy 3, Cy3.5 and Cy5 etc.), which all have unique Raman scattering peak that not overlapping with others. Immobilization of such Raman dyes has been well studied and is technically feasible. With different dyes labeling each analyte-specific metallic particle that bond with the corresponding antibody, there will be several different kinds of particle clusters formed for the multiplexed AMSERSIA assay as shown in FIG. 4. The concentration of each analyte can be determined by the unique Raman scattering peaks of the corresponding Raman dye.

Examples will now be given.

1) Synthesis of Mono-Dispersed Gold Nano-Particles

9 mL of 10⁻⁴ M aqueous solution of chloroauric acid (HAuCl4) is reduced by 1 mL of 10⁻³ M aqueous solution of aspartic acid under boiling condition for about 10-15 minutes to yield ruby-red color solution. The mono-dispersed gold nano-particle size is around 25 nm by our TEM analysis. The gold particle size can be varied via changing the concentration of aspartic acid solution.

2) Modification of Nano-Particle with Mercapto-Carboxylic Acid

To the 1 mL above gold particle solution add 100 μL of 50 mM of mercaptoacetic acid (in borate buffer, pH 9, 50 mM). The solution is stirred at room temperature for about 2-3 hours, and the modified gold particle is washed either by centrifugation or dialysis with HEPES buffer (pH 7.5, 50 mM) to remove excess free mercaptoacetic acid.

3) Coupling of Antibody to Gold Particle

The surface modified gold particle (1 mL) is mixed with certain amount Ab (based on the assay requirement) at room temperature for about 30 minutes. Then, about 100 μL of 500 mM EDAC is added into the solution with stirring. The solution is stirred gently at room temperature overnight. Large amount BSA (100 μL of 100 mg/mL BSA solution) is then added into the solution to block the non-specific binding site of the gold particle. The antibody coupled gold particle is then washed with either centrifugation or dialysis with PBS buffer (50 mM, pH 7.4 with 0.2% Tween-20).

4) Coupling of Raman Dye to the Particle Surface.

To the antibody modified gold particle solution add amino-reactive functionalized dyes (containing NHS ester, isothiocynate or aldehyde etc groups), which are either commercially available (Cy-NHS ester dyes or Rhodamine isothiocyanate) or made in the lab. The Raman dye is usually dissolved into DMSO or DMF solution at about 10 mg/mL to 100 mg/mL concentration depending on the individual assay. The dye solution is then added into the gold particle solution (in PBS buffer with about 0.2% Tween-20) and stirred at room temperature over night. The dye modified gold particle is then washed either with centrifugation or dialysis bag to remove un-bounded dye. The washed gold particle is diluted to certain concentration into the assay buffer and ready for assay test.

5) Typical Assay Condition and Sensitivity

For AFP assay (alpha Fetoprotein), we use PBS (50 mM, pH 7.4) with 0.2% Tween-20 as our assay buffer. The gold particle concentration is typically around 500 μg/mL. We add about 90 μL of gold particle solution 1 and 90 μL of gold particle solution 2 into the test cuvette, and then add about 10 μL of sample and another 110 μL of assay buffer. The assay mixture is incubated at 37° C. for about 25 minutes. Then collect the Raman signal. The AFP level is calculated from the standard curve, which is calibrated with 5 level calibrators (0 ng/mL, 2 ng/mL, 50 ng/mL, 300 ng/mL and 1000 ng/mL). The detection limit of this assay is about 0.5 ng/mL.

While some preferred embodiments of the present invention have been set forth in detail, those skilled in the art who have reviewed the present disclosure will readily appreciate that other embodiments can be realized within the scope of the invention. For example, disclosures of any particular analytes, reagents, nanoparticles, or Raman dyes are illustrative rather than limiting, as are disclosures of concentrations or other numerical values. Therefore, the present invention should be construed only by the appended claims.

1. A method for detecting an analyte, the method comprising:
a. providing a group of metallic nano-particles, each particle being bonded with at least one antibody molecule and a Raman dye;
b. causing molecules of the analyte to react with the antibody molecules bonded on individual ones of the metallic particles and binding two or more of the metallic particles together to form a cluster structure; and
c. detecting the Raman dye on the cluster structure through a Raman scattering signal, wherein a proximity effect of the cluster structure amplifies the Raman scattering signal.

2. The method of claim 1, wherein the metallic nano-particles comprise a metal selected from the group consisting of silver and gold.

3. The method of claim 1, wherein step (b) comprises causing a molecule of the analyte to react with the antibody molecules bonded on at least two of the metallic particles to form a sandwich structure.

4. A method for detecting an analyte, the method comprising:
a. providing a group of metallic nano-particles, each particle being bonded with at least one analyte analogue molecule and a Raman dye;
b. exposing the metallic particles to antibody molecules to cause the antibody molecules to react with the analyte analogue molecules bonded to individual ones of the metallic particles and binding two or more of the metallic particles together to form a cluster structure;
c. causing the antibody molecules to react with molecules of the analyte to inhibit a formation of said cluster structure; and
d. detecting the Raman dye on the cluster structure through a Raman scattering signal, wherein a proximity effect of the cluster structure amplifies the Raman scattering signal.

5. The method of claim 4, wherein the metallic nano-particles comprise a metal selected from the group consisting of silver and gold.

6. The method of claim 4, wherein step (b) comprises causing an antibody molecule to react with the analyte analogue molecules bonded on at least two of the metallic particles to form a sandwich structure.

7. A method for detecting an analyte which is one of a plurality of analytes, the method comprising:
a. providing a plurality groups of metallic nano-particles, each group of particles being bonded with unique Raman dyes having different Raman spectra and antibody molecules specific to one of the plurality of analytes;
b. causing molecules of the analyte to react with the specific antibody molecules bonded to corresponding ones of the metallic particles and binding the a plurality of the corresponding ones of the metallic particles together to form a cluster structure;
c. detecting the Raman dyes on the cluster structure through a Raman scattering signal; and
d. identifying the analyte from the plurality of analytes accordance with a Raman spectrum of the Raman scattering signal, wherein a proximity effect of the cluster structure amplifies the Raman scattering signal.

8. The method of claim 7, wherein the metallic nano-particles comprise a metal selected from the group consisting of silver and gold.

9. The method of claim 8, wherein step (b) comprises causing a molecule of the analyte to react with the antibody molecules bonded on at least two of the metallic particles to form a sandwich structure.

10. The method of claim 8, wherein steps (c) and (d) are performed a plurality of times to detect different analytes.
